(19) United States
(12) Patent Application Publication
(10) Pub. No.: US 2013/0189707 A1
(43) Pub. Date: Jul. 25, 2013

(54) METHOD OF DETERMINATION OF AUTOANTIBODY LEVEL BY MEANS OF ENZYME IMMUNOASSAY

(71) Applicants: Svetlana Alexandrovna Sergeeva, Moscow (RU); Sergei Alexandrovich Tarasov, Odintsovsky r-n (RU); Alexander Vladimirovich Tarasov, Odintsovsky r-n (RU); Peter H. Van Der Meide, Maarn (NL)

(72) Inventors: Svetlana Alexandrovna Sergeeva, Moscow (RU); Sergei Alexandrovich Tarasov, Odintsovsky r-n (RU); Alexander Vladimirovich Tarasov, Odintsovsky r-n (RU); Peter H. Van Der Meide, Maarn (NL)

(21) Appl. No.: 13/575,320

(22) PCT Filed: Jan. 24, 2011
(86) PCT No.: PCT/RU11/00034

§ 371 Date: Feb. 8, 2013

(30) Foreign Application Priority Data
May 5, 2010 (RU) 2010117620
Jan. 26, 2011 (RU) 201012114

(51) Int. Cl. G01N 33/543

(52) U.S. Cl. CPC G01N 33/54393
USPC 435/7.5

(57) ABSTRACT

The method for quantitative determination of the level of natural autoantibodies in human biological fluids, when as a solid phase of physical sorption is used the solid phase of physical sorption, coated with streptavidin, and the solid phase of physical sorption is treated with preliminary biotinylated antigen and blocking agent for closing the sites of nonspecific binding at the solid phase of physical sorption, for which purpose are used proteins, biotinylated according to standard procedure. As the conjugate-containing solution are used enzyme-labeled monoclonal and polyclonal antibodies, which react with one or all isotypes of human immunoglobulins. In addition, the tested biological fluid is preliminary diluted in a buffer, containing proteins which are used for closing the sites of nonspecific binding at solid phase of physical sorption, and also substances protecting natural autoantibodies from destruction during heat treatment, and subjected to heat treatment. For each tested specimen of biological fluid, a control solid phase of physical sorption is used, and the number of natural autoantibodies is determined with the use of a calibration curve which is plotted using monoclonal or polyclonal antibodies to antigen.

### TECHNICAL FIELD

The invention is related to the field of medicine, in particular to laboratory diagnostics, and it can be used to improve efficiency and reliability of quantitative determination of natural autoantibody concentration in human biological fluids.

### PREVIOUS TECHNICAL LEVEL

There is a known technical method for quantitative determination of autoantibody level to endogenous proteins in human biological fluids by means of solid phase enzyme immunoassay (EIA) (RU 2137134 C1, G01N33/53, 1999). However, this method is of little use for the quantitative determination of natural autoantibody level in human biological fluids.

A method of enzyme immunoassay is known, which includes adsorption of antigens at a solid phase of physical sorption, incubation of tested biological specimens, incubation of a conjugate-containing solution, and spectrophotometric analysis of the reaction of extinction of a chromatic agent (RU 2014610 C1, G01N33/535, 1994).

In addition, there is a known method to perform an enzyme immunoassay for determining the level of natural autoantibodies which includes adsorption of antigens at a solid phase of physical sorption, incubation of tested biological specimens, incubation of a conjugate-containing solution, and spectrophotometric analysis of the reaction of extinction of a chromatic agent (BMS217TEN Human anti-IFN-alpha ELISA, Bender MedSystems GmbH, Austria). Despite sufficient simplicity, sensitivity and specificity of this solution, the obtained results can be false positive because of nonspecific and low-affinity binding of various serum or plasma immunoglobulins with antigen adsorbed on EIA plate. Besides the presence in blood of polyreactive immunoglobulins not belonging to the pool of natural antibodies can cause false-positive results because polyreactive immunoglobulins non-specifically binds to components of the EIA plate. In addition, only the free natural antibody fraction is detected in EIA, while the most part of natural autoantibodies are present in blood in a complex, bound with its antigen.

### DISCLOSURE OF INVENTION

The invention is intended to develop a technologically simple, sensitive and specific method for quantitative determination of natural autoantibody level in human biological fluids using the EIA method.

The set task is solved due to the fact that in the method of quantitative determination of natural autoantibody level in human biological fluids with the aid of the enzyme immunoassay, including the antigen treatment of physical sorption solid phase, addition of tested biological specimens, solid phase treatment with a conjugate-containing solution, separation of solid and liquid phases, and spectrophotometric analysis of a reaction for extinction of a chromatic agent solution, according to the invention, the physical sorption solid phase, coated with streptavidin, is used as the physical sorption solid phase, and the physical sorption solid phase is treated with the pre-biotinylated antigen and blocking agent for closing the sites of nonspecific binding at the physical sorption solid phase, for which purpose are used proteins biotinylated according to the standard procedure. As a conjugate-containing solution are used monoclonal or polyclonal enzyme--labeled antibodies which react with one or all isotypes of human immunoglobulins. In addition, the tested biological fluid is preliminary diluted in a buffer, containing proteins which are used to close the sites of nonspecific binding at the physical sorption solid phase, and also substances which protect natural autoantibodies from destruction during heat treatment, and subject it to heat treatment. For each tested specimen of biological fluid, a corresponding control physical sorption solid phase is used, at which the biotinylated antigen is not immobilized (i.e. for each tested specimen of biological fluid for binding with the solid phase streptavidin, a biotinylated control protein is used which is similar to protein used for closing the nonspecific binding sites at physical sorption solid phase, which makes it possible to measure spectrophotometric signal, specific and nonspecific for natural autoantibodies), and the number of natural autoantibodies is determined with the aid of a calibration curve which is standardized over monoclonal or polyclonal antibodies to antigen.

In addition, before heat treatment, the tested biological fluid, diluted in a buffer containing proteins which are used for closing the nonspecific binding sites at the physical sorption solid phase, and substances protecting natural autoantibodies from destruction during thermal treatment, are additionally treated with an iron-containing oxidizer.

Due to combined application of the physical sorption solid phase, coated with streptavidin, and biotinylated agents at the physical sorption solid phase, all epitopes of the immobilized antigen remain free for binding with natural autoantibodies, thus increasing sensitivity of the claimed method, while direct immobilization of an antigen results in conformational changes and destruction of antigen epitopes, which reduces sensitivity of EIA method. In addition, application of enzyme--labeled antibodies reacting with one or all isotypes of human immunoglobulins, makes it possible to determine the level of a certain isotype or all isotypes of natural autoantibodies and, respectively, extends functional capabilities of the claimed method. Preliminary dilution of the investigated specimen in a buffer, containing proteins which are present in the blocking buffer and are used for closing the sites of nonspecific binding at physical sorption solid phase, minimizes the possibility of nonspecific binding the antibodies of the investigated specimen with the solid phase, at which proteins are immobilized, which also increases specificity and sensitivity of the claimed method.

Addition of iron-containing oxidizer and heat treatment of the investigated specimen makes it possible to destroy the complex of natural autoantibodies with antigens and anti-idiotypic antibodies, or to ensure availability of antigen paratopes to antigen epitopes due to various demasking effects, and preliminary dilution of the investigated specimen in a buffer protects natural autoantibodies from destruction during heat treatment, which allows to determine the total level of natural autoantibodies (i.e. as free and antigen-bounded forms of natural autoantibodies or natural antibodies with closed paratopes), and thus reduces the possibility of obtaining the false negative results, increases sensitivity, efficiency and functional capabilities of the claimed method.

Usage for each investigated specimen of biological fluid individual control solid phase of the physical sorption with when immobilized biotinylated blocking protein, makes it possible to estimate specific and nonspecific for natural autoantibodies, spectrophotometric signal, which also reduced the possibility of obtaining false positive results and increased sensitivity of the claimed method.

In addition, the claimed combination of essential features, which extends the possibility of quantitative determination of the level of natural autoantibodies, extends the range of technical means for solving the set task.

### SHORT DESCRIPTION OF DRAWING FIGURES

FIG. 1 shows the calibration curve for example 1;

FIG. 2 shows the calibration curve for example 2;

FIG. 3 shows the calibration curve for example 3.

### INVENTION EMBODIMENTS

The claimed method for qualitative determination of the level of natural autoantibodies in human biological fluids has been implemented as follows:

Solid phase of physical sorption (wells) of a standard well plate is coated with streptavidin or its analogs (for example, avidin, etc), the physical sorption solid phase is blocked with a blocking agent and incubated with a biotinylated agent to which it is needed to determine the level of natural autoantibodies, or (control wells) with biotinylated blocking protein (for example, bovine serum albumin, ovalbumin, human serum albumin, rabbit serum albumin, gelatin, etc), which is the control of nonspecific binding. Antigens are biotinylated by the minimum quantity of biotin (for example, D-biotinoyl-E-aminocaproic acid-N-hydroxysuccinimide ester) (1:2 antigen to biotin molar ratio) to minimize epitope destruction.

Biological fluid is prepared for the assay (for example, blood serum, blood plasma, etc) by dilution (in 50 to 200,000 time range) in a buffer solution which contains proteins in the composition of the blocking buffer (for example, bovine serum albumin, ovalbumin, human serum albumin, rabbit serum albumin, gelatin, etc), preservatives (for example, timerosal, etc), surface active substances (for example, Triton-X100, etc), which protect natural autoantibodies from destruction during heat treatment , and the obtained solution of biological fluid is heat treated within the temperature range of 50° C. to 80° C.

It is recommended that prior to heat treatment the tested biological fluid shall be treated additionally with an iron-containing oxidizer (for example, ferric chloride (III) [FeCl₃]).

Then the heat treated solution of the investigated biological fluid is placed into wells the same as specific antibodies used for plotting a calibration curve, for which purpose are used monoclonal or polyclonal antibodies to the antigen with the known concentration. In order to reveal the formed immune complex (antigen-natural antibodies), the conjugate of polyclonal or monoclonal antibodies (for example, goat polyclonal antibodies, sheep polyclonal antibodies, mice monoclonal antibodies, etc) to light chains of a certain or all human immunoglobulins with an enzyme (for example, alkaline phosphatase, horseradish peroxidase, etc) are used. The level of natural autoantibodies is determined with respect to the reaction of extinction of chromatic agent solution which changes its color depending on the quantity of chromatic agent isolated from the substrate upon its decomposition by an enzyme (form example, alkaline phosphatase, etc); the substrate produces a soluble product whose color characteristics can be measured spectrophotometrically at a certain wavelength.

### EXAMPLE 1

In order to confirm the possibility of obtaining the technical effect while implementing the claimed method of quantitative determination of the level of natural autoantibodies to human gamma interferon, serum specimens were taken from twenty healthy donors who were not subjected to interferon therapy. Hence, their serum does not contain autoantibodies to human gamma interferon, yet natural autoantibodies to human gamma interferon are present.

A portion of wells of 96-well plate, coated with streptavidin from Greiner bio-one GmbH (Catalog [symbol] 655990), were inoculated with 100 μl of the recombinant human gamma interferon from eBiosciences (Catalog [symbol] 34-8319-85), and the other portion (control wells for investigated specimens) were inoculated with bovine serum albumin from Sigma Aldrich (Catalog [symbol] A-3803), both biotinylated according to the standard procedure of U-CyTech Bioscience Company (Standard Operating Procedure [symbol] UCT-127) at the rate of 100 μl/well at 100% humidity (the principle of the labeling is that free amino groups of the proteins react with D-biotinoyl-ε-aminocaproic acid-N-hydroxysuccinimide ester (NHS-biotin) (1:2 antigen to biotin molar ratio) by forming a stable amide bond (the reaction takes place at room temperature within 2h incubation under gentle stirring); the biotinylated protein is then dialysed against phosphate buffer solution to remove non-reacted NHS-biotin).

Blood serum specimens were diluted by 20 times (100 μl of serum were diluted in 1900 μl of phosphate buffer containing 1% Triton-X100 and 0.002% timerosal), and were incubated for 20 minutes at 75° C.

Then the incubated solution was again diluted by 5 times (100 μl of incubated solution were diluted in 400 μl of phosphate buffered saline containing 1% bovine serum albumin (BSA), 1% Triton-X100 and 0.002% timerosal), and was further used for inoculation in the plate wells (100 μl per well).

In order to plot a calibration curve, standard antibody solutions were prepared (mice monoclonal antibodies to human gamma interferon, clone MD-2) within the concentration range from 16 units/ml to 0.5 units/ml. 100 picogram/ml of murine monoclonal antibodies was arbitrarily defined as 1 Unit/ml.

The prepared standard antibodies, the solution used as negative control (phosphate buffer containing 1% BSA, 1% Triton-X100 and 0.002% timerosal) and solutions of the investigated specimens were inoculated into plate wells as shown in Table 1, and incubated at 37° C. for 2 hours. After incubation, decanting was used to remove fluid from the plate, and the plate was rinsed with a standard washing solution (phosphate buffer containing 0.05% Twin-20 and 0.01% timerosal).

Table 1 shows the chart of specimen inoculation into plate wells.

TABLE 1

| | I | | II | | III | | IV | | V | | VI | |
|---|---|---|---|---|---|---|---|---|---|---|---|---|
| | Biotin- | Biotin- | Biotin- | Biotin- | Biotin- | Biotin- | Biotin- | Biotin- | Biotin- | Biotin- | Biotin- | Biotin- |
| Immobi- | ylated | ylated | ylated | ylated | ylated | ylated | ylated | ylated | ylated | ylated | ylated | ylated |
| lized | IFN-γ | IFN-γ | IFN-γ | BSA | IFN-γ | BSA | IFN-γ | BSA | IFN-γ | BSA | IFN-γ | BSA |
| antigen | 1 | 2 | 3 | 4 | 5 | 6 | 7 | 8 | 9 | 10 | 11 | 12 |
| A | P1 | P1 | S1 | S1 | S5 | S5 | S9 | S9 | S13 | S13 | S17 | S17 |
| B | P2 | P2 | S1 | S1 | S5 | S5 | S9 | S9 | S13 | S13 | S17 | S17 |
| C | P3 | P3 | S2 | S2 | S6 | S6 | S10 | S10 | S14 | S14 | S18 | S18 |
| D | P4 | P4 | S2 | S2 | S6 | S6 | S10 | S10 | S14 | S14 | S18 | S18 |
| E | P5 | P5 | S3 | S3 | S7 | S7 | S11 | S11 | S15 | S15 | S19 | S19 |
| F | P6 | P6 | S3 | S3 | S7 | S7 | S11 | S11 | S15 | S15 | S19 | S19 |
| G | N | N | S4 | S4 | S8 | S8 | S12 | S12 | S16 | S16 | S20 | S20 |
| H | N | N | S4 | S4 | S8 | S8 | S12 | S12 | S16 | S16 | S20 | S20 |

Comment:
P1: Standard antibodies (16 units/ml)
P2: Standard antibodies (8 units/ml)
P3: Standard antibodies (4 units/ml)
P4: Standard antibodies (2 units/ml)
P5: Standard antibodies (1 unit/ml)
P6: Standard antibodies (0.5 units/ml)
N: Negative control - phosphate buffer, containing 1% BSA, 1% Triton-X100 and 0.002% timerosal
S1-S20: Investigated samples 1-20
IFN-γ - recombinant human gamma interferon
BSA - bovine serum albumin

Then, all plate wells were inoculated with 100 μl each, containing a mixture of goat antibodies to human IgA, IgM, IgG (Sigma Aldrich; Catalog [symbol] A-3313), conjugated with alkaline phosphatase, which makes it possible to determine the level of all isotypes of natural autoantibodies to human gamma interferon, or goat antibodies to mice IgG (Sigma Aldrich; Catalog [symbol] A-3562), conjugated with alkaline phosphatase, for determining the level of monoclonal antibodies to human gamma interferon (MD-2), used for plotting the calibration curve. Then the plate was incubated for 1 hour at 37° C. After incubation, fluid was removed from the plate wells by decanting, and the plate was rinsed 5 times with a standard washing solution (a phosphate buffer containing 0.05% Twin-20 and 0.01% timerosal).

15 minutes prior to the end of incubation with conjugate-containing solution, a chromatic agent solution was prepared by dissolving 1 tablet of a substrate buffer and 1 tablet of substrate (para-nitrophenylphosphate) from Sigma Company (Catalog [symbol] N-2770) in 20 ml of distilled water.

100 μl of the prepared solution of the chromatic agent were inoculated into each plate well, and the plate was incubated for 30 minutes at 37° C.

After incubation, all plate wells were inoculated with a stopping solution (3N sodium hydroxide) from Merck KgaA (Catalog [symbol] 1.06495), and extinction was measured at wavelength of 405 nm, using a standard photometer.

The obtained results of measurements were used to calculate the level of natural autoantibodies to gamma interferon. For this purpose, true value of optical density (OD) of standard antibodies and true values of investigated specimens was determined as follows:
1. The mean OD for negative control (the mean arithmetic value of OD in wells 1G, 1H, 2G, 2H) was calculated.
2. The mean OD for standard antibodies (the mean arithmetic value of OD in wells of the respective standard P1-P6) was calculated.
3. True value of OD of standard antibodies was calculated as the difference of the mean value of OD of standard antibodies and the mean value of OD of negative control. Results of calculations are given in Table 2.

TABLE 2

| | | | | | St. antibody OD |
|---|---|---|---|---|---|
| | | | | OD mean | minusmean OD of |
| | | 1 | 2 | values: | negative control: |
| P1 | A | 1.704 | 1.554 | 1.629 | 1.521 |
| P2 | B | 1.122 | 1.202 | 1.162 | 1.054 |
| P3 | C | 0.818 | 0.801 | 0.810 | 0.702 |
| P4 | D | 0.497 | 0.453 | 0.475 | 0.367 |
| P5 | E | 0.285 | 0.266 | 0.276 | 0.168 |
| P6 | F | 0.201 | 0.188 | 0.195 | 0.087 |
| N | G | 0.115 | 0.108 | 0.108 | |
| N | H | 0.106 | 0.102 | | |

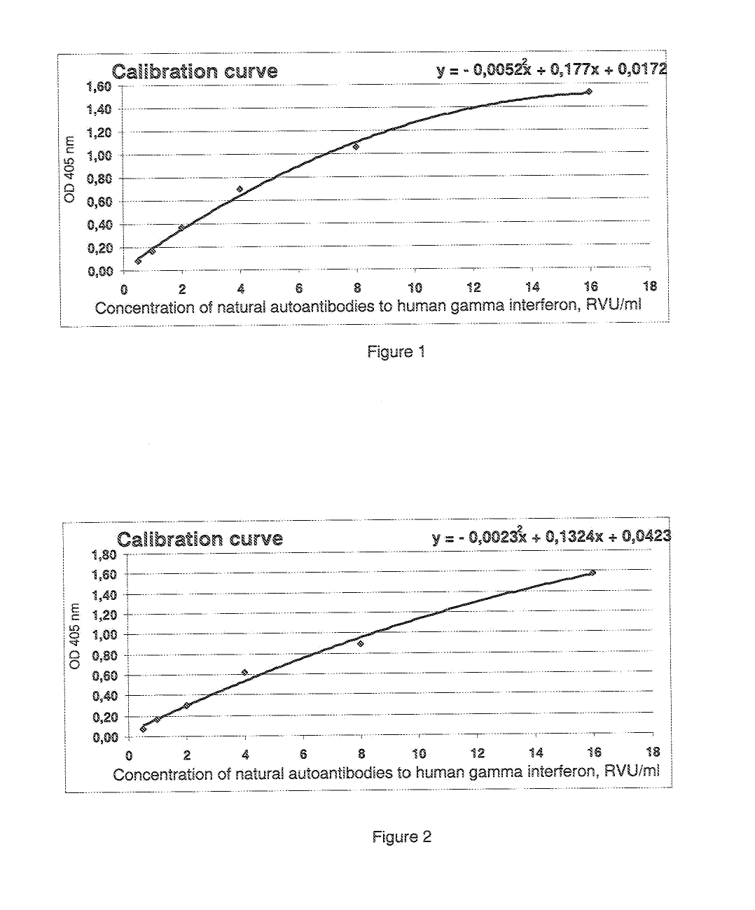

4. The obtained values of true OD of standard antibodies were used to plot the calibration curve (see FIG. 1), where OD values are on the axis of ordinates (y), and on the axis of abscissas (x) is concentration of antibodies to gamma interferon. The plotted calibration curve can be described by the following equation: y=−0.0052x²+0.177x+0.0172
5. The mean OD was calculated for the investigated specimens in plate wells in which biotinylated IFN-γ (rows 3,5,7,9,11 in Table 1) is immobilized, and in which biotinylated BSA (rows 4,6,8,10,12 in Table 1) is immobilized. For example, for the investigated specimen S1, the mean OD value was calculated in wells A3, B3 and A4. B4, respectively 6. OD true value was calculated for the investigated specimens as the difference of the OD mean value for the investigated specimens measured in plate wells, where biotinylated IFN-γ is immobilized, and OD mean value for the investigated specimens measured in plate wells, where biotinylated BSA is immobilized. For example, for the investigated specimen S1, from the OD mean value, measured in plate wells A3, B3, the OD mean value, measured in plate wells A4, B4, is deducted.
7. The calibration curve (FIG. 1) was used to determine the concentration of diluted natural autobodies to human gamma interferon in the investigated specimens. In order to obtain the true value of concentration of natural autoantibodies to human gamma interferon in the investigated specimens, the obtained result was multiplied by the degree of specimen dilution (by 100).
8. The results are presented in Table 3.

TABLE 3

| Investigated | Concentration of natural autoantibodies |
|---|---|
| specimen | to human gamma interferon, Unit/ml |
| S1 | 780.1 |
| S2 | 774.6 |
| S3 | 288.3 |
| S4 | 977.1 |
| S5 | 509.0 |
| S6 | 688.1 |
| S7 | 794.5 |
| S8 | 572.4 |
| S9 | 453.5 |
| S10 | 753.1 |
| S11 | 527.9 |
| S12 | 298.8 |
| S13 | 345.6 |
| S14 | 443.5 |
| S15 | 621.8 |
| S16 | 575.2 |
| S17 | 758.9 |
| S18 | 857.5 |
| S19 | 656.2 |
| S20 | 753.5 |

### EXAMPLE 2

Serum specimens were taken from twenty patients with infectious mononucleosis, at which the level of natural autoantibodies to human gamma interferon increases.

All stages of determining the level of natural autoantibodies to human gamma interferon were similar to those described in Example 1, except for the fact that the investigated specimens were diluted by 1000 times, human serum albumin was used as a blocking agent, and the incubation temperature was 56° C.

The calibration curve is shown in FIG. 2, and the obtained results are presented in Table 4.

TABLE 4

| Investigated | Concentration of natural autoantibodies |
|---|---|
| specimen | to human gamma interferon, RVU/ml |
| S1 | 1675.9 |
| S2 | 1591.7 |
| S3 | 1897.5 |
| S4 | 1480.0 |
| S5 | 1924.4 |
| S6 | 1974.6 |
| S7 | 1537.4 |
| S8 | 1221.1 |
| S9 | 1624.5 |
| S10 | 1569.9 |
| S11 | 1874.6 |
| S12 | 1530.2 |
| S13 | 1870.8 |
| S14 | 1798.9 |
| S15 | 2151.8 |
| S16 | 1882.2 |
| S17 | 1505.0 |
| S18 | 1810.2 |
| S19 | 1526.6 |
| S20 | 1451.4 |

### EXAMPLE 3

To verify the possibility of obtaining the technical effect when implementing the claimed method for quantitative determination of the level of natural autoantibodies to human gamma interferon, where prior to heat treatment, the tested biological fluid is additionally treated with iron-containing oxidizer, specimens were taken from twenty healthy donors who were not subjected to interferon therapy and, therefore, there is no autoantibodies to human gamma interferon in serum, yet there are natural autoantibodies to human gamma interferon.

A portion of wells of 96-well plate, coated with streptavidin from Greiner bio-one GmbH (Catalog [symbol] 655990), were inoculated with 100 μl of the recombinant human gamma interferon from eBiosciences (Catalog [symbol] 34-8319-85), and the other portion (control wells for investigated specimens) were inoculated with bovine serum albumin from Sigma Aldrich (Catalog [symbol] A-3803), both biotinylated according to the standard procedure of U-CyTech Bioscience Company (Standard Operating Procedure [symbol] UCT-127) at the rate of 100 μl/well at 100% humidity (the principle of the labeling is that free amino groups of the proteins react with D-biotinoyl-E-aminocaproic acid-N-hydroxysuccinimide ester (NHS-biotin) by forming a stable amide bond (the reaction takes place at room temperature within 2 h incubation under gentle stirring); the biotinylated protein is then dialysed against phosphate buffer solution to remove non-reacted NHS-biotin).

Blood serum specimens were diluted by 10 times (10 ρl of serum were diluted in 90 μl of a phosphate buffer containing 1% Triton-X100 and 0.002% timerosal), treated with 2 mM FeCl₃ and incubated for 40 minutes at 56° C.

Then the incubated solution was diluted additionally by 5 times (100 μl of incubated solution were diluted in 400 μl of a phosphate buffer containing 1% bovine serum albumin (BSA),1% Triton-X100 and 0.002% timerosal), and it was further used for inoculating into the plate wells (100 μl per well).

In order to plot the calibration curve, solutions of standard antibodies (mice monoclonal antibodies to human gamma interferon, clone MD-2) were prepared within the range of concentrations from 16 Units/ml to 0.5 Units/ml. 100 picogram/ml of murine monoclonal antibodies was arbitrarily defined as 1 Unit/ml.

The prepared standard antibodies, a solution used as negative control (a phosphate buffer containing 1% BSA, 1% Triton-X100 and 0.002% timerosal), and solutions of the investigated specimens were inoculated into plate wells as shown in Table 5, and they were incubated at 37° C. for 2 hours. After incubation, fluid was removed from the plate by decanting, and the plate was rinsed 5 times with a standard washing solution (a phosphate buffer containing 0.05% Twin-20 and 0.01% timerosal).

Table 5 shows the chart for placing specimens into the plate wells.

TABLE 5

| | I | | II | | III | | IV | | V | | VI | |
|---|---|---|---|---|---|---|---|---|---|---|---|---|
| | Biotin- | Biotin- | Biotin- | Biotin- | Biotin- | Biotin- | Biotin- | Biotin- | Biotin- | Biotin- | Biotin- | Biotin- |
| Immobi- | ylated | ylated | ylated | ylated | ylated | ylated | ylated | ylated | ylated | ylated | ylated | ylated |
| lized | IFN-γ | IFN-γ | IFN-γ | BSA | IFN-γ | BSA | IFN-γ | BSA | IFN-γ | BSA | IFN-γ | BSA |
| antigen | 1 | 2 | 3 | 4 | 5 | 6 | 7 | 8 | 9 | 10 | 11 | 12 |
| A | P1 | P1 | S1 | S1 | S5 | S5 | S9 | S9 | S13 | S13 | S17 | S17 |
| B | P2 | P2 | S1 | S1 | S5 | S5 | S9 | S9 | S13 | S13 | S17 | S17 |
| C | P3 | P3 | S2 | S2 | S6 | S6 | S10 | S10 | S14 | S14 | S18 | S18 |
| D | P4 | P4 | S2 | S2 | S6 | S6 | S10 | S10 | S14 | S14 | S18 | S18 |
| E | P5 | P5 | S3 | S3 | S7 | S7 | S11 | S11 | S15 | S15 | S19 | S19 |
| F | P6 | P6 | S3 | S3 | S7 | S7 | S11 | S11 | S15 | S15 | S19 | S19 |
| G | N | N | S4 | S4 | S8 | S8 | S12 | S12 | S16 | S16 | S20 | S20 |
| H | N | N | S4 | S4 | S8 | S8 | S12 | S12 | S16 | S16 | S20 | S20 |

Comment:
P1: Standard antibodies (16 units/ml)
P2: Standard antibodies (8 units/ml)
P3: Standard antibodies (4 units/m)
P4: Standard antibodies (2 units/ml)
P5: Standard antibodies (1 units/ml)
P6: Standard antibodies (0.5 units/ml)
N: Negative control - a phosphate buffer containing 1% BSA, 1% Triton-X100 and 0.002% timerosal
S1-S20: Investigated specimens 1-20
IFN-γ - recombinant human gamma interferon
BSA - bovine serum albumin

Then, all plate wells were inoculated with 100 μl of solution, containing a mixture of goat antibodies to human IgA, IgM, IgG (Sigma Aldrich; Catalog. [symbol] A-3313), conjugated with alkaline phosphatase, which allows to determine the level of all isotypes of natural autoantibodies to human gamma interferon, or goat antibodies to mice IgG (Sigma Aldrich; Catalog [symbol] A-3562), conjugated with alkaline phosphatase, for determining the level of monoclonal antibodies to human gamma interferon (MD-2), used for plotting a calibration curve. Then the plate was incubated for 1 hour at 37° C. After incubation, fluid was removed from plate wells by decanting, and the plate was rinsed 5 times with a washing solution (a phosphate buffer containing 0.05% Twin-20 and 0.01% timerosal).

15 minutes before the end of incubation with conjugate-containing solution, a chromatic agent solution was prepared by dissolving 1 tablet of a substrate buffer and 1 tablet of a substrate (para-nitrophenylphosphate) from Sigma Company (Catalog [symbol] N-2770) in 20 ml of distilled water.

100 μl of the prepared chromatic agent solution were placed into each plate well, and the plate was incubated for 30 minutes at 37° C.

After incubation, all plate wells were inoculated with the stopping solution (3N sodium hydroxide) from Merck KgaA (Catalog [symbol] 1.06495), and extinction was measured at 405 nm wavelength using a standard photometer.

The obtained measurement results were used to calculate the level of natural autoantibodies to gamma interferon. For this purpose, the true value of optical density (OD) of standard antibodies and the true value of OD of the investigated specimens were determined as follows:
1. The mean OD was calculated for negative control (the mean arithmetic OD value in wells 1G, 1H, 2G, 2H)
2. The mean OD was calculated for standard antibodies (the mean arithmetic OD value in wells of the respective standard P1-P6).
3. The true value of OD of standard antibodies was calculated as the difference of the OD mean value of standard antibodies and the mean OD value of negative control. The results of calculations are presented in Table 6.

TABLE 6

| | | | | | Mean OD of st. |
|---|---|---|---|---|---|
| | | | | OD mean | antibodies, minus mean |
| | | 1 | 2 | values: | OD of negative control: |
| P1 | A | 1.798 | 1.781 | 1.790 | 1.657 |
| P2 | B | 1.253 | 1.311 | 1.282 | 1.149 |
| P3 | C | 0.806 | 0.744 | 0.775 | 0.642 |
| P4 | D | 0.509 | 0.577 | 0.543 | 0.410 |
| P5 | E | 0.284 | 0.338 | 0.311 | 0.178 |
| P6 | F | 0.219 | 0.235 | 0.227 | 0.094 |
| N | G | 0.138 | 0.125 | 0.133 | |
| N | H | 0.139 | 0.129 | | |

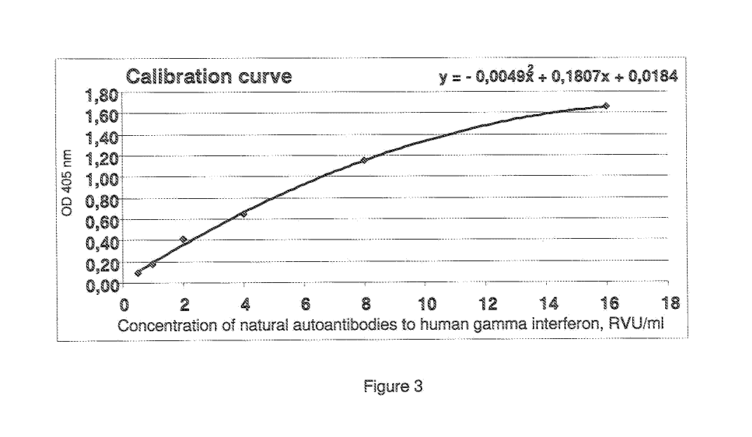

4. The obtained values of the true OD of standard antibodies were used to plot a calibration curve (see FIG. 3), where on the axis of ordinates (y) are OD values, and of the axis of abscissas (x) is the concentration of antibodies to gamma interferon. The plotted calibration curve can be described by the following equation: y=−0.0049x2+0.1807x+0.0184
5. The mean OD for the investigated specimens was calculated in plate wells in which biotinylated IFN-γ (rows 3,5,7,9,11 in Table 5) is immobilized, and in which biotinylated BSA (rows 4,6,8,10,12 in Table 5) is immobilized. For example, for the investigated specimen S1, the mean OD value in wells A3, B3 and A4, B4, respectively.
6. The true OD value for the investigated specimens was calculated as the difference of the mean OD value for the investigated specimens, measured in plate wells, where biotinylated IFN-γ is immobilized, and the mean OD value in which biotinylated SA is immobilized. For example, for the investigated specimen S1, the mean OD value, measured in plate wells A4, B4, is deducted from the mean OD value, measured in plate values A3, B3.
7. Using the calibration curve (FIG. 3), concentration of diluted natural autoantibodies to human gamma interferon was determined in the investigated specimens. To obtain the true value of concentration of natural autoantibodies to human gamma interferon in the investigated specimens, the obtained results was multiplied by the degree of dilution of the specimens (by 100).
8. The results are presented in Table 7.

TABLE 7

| Investigate | Concentration of natural autoantibodies |
|---|---|
| specimen | to human gamma interferon, RVU/ml |
| S1 | 744.4 |
| S2 | 935.6 |
| S3 | 876.8 |
| S4 | 563.9 |
| S5 | 1001.1 |
| S6 | 957.6 |
| S7 | 1019.1 |
| S8 | 794.7 |
| S9 | 592.5 |
| S10 | 1088.8 |
| S11 | 1116.4 |
| S12 | 1006.8 |
| S13 | 1036.6 |
| S14 | 428.0 |
| S15 | 422.2 |
| S16 | 1433.9 |
| S17 | 1042.9 |
| S18 | 1354.5 |
| S19 | 702.5 |
| S20 | 1344.2 |

Thus, the presented examples of application of blood serum, containing natural autoantibodies to human gamma interferon, prove efficiency and reliability of quantitative determination of the level of natural autoantibodies to human gamma interferon upon implementation of the claimed combination of features. Also, additional treatment of the tested biological fluid increases efficiency of the claimed method which follows from Example 3.

1. A method for quantitative determination of natural autoantibodies in human biological fluids by enzyme immunoassay, comprising treatment of the solid phase of physical sorption with antigen, addition of tested biological specimens, treatment of the solid phase with a conjugate-containing solution, separation of solid and liquid phases, and spectrophotometric analysis of the reaction by extinction of a chromatic agent solution.

2. (canceled)

3. The method of claim 1 wherein the solid phase of physical sorption is coated with streptavidin, and the solid phase of physical sorption is treated with pre-biotinylated antigen and a blocking agent.

4. The method of claim 1 wherein the conjugate-containing solution comprises monoclonal and polyclonal antibodies, labeled with an enzyme and reacting with one or all isotypes of human immunoglobulins.

5. The method of claim 1 wherein the biological fluid is preliminary diluted in a buffer, containing proteins and substances, protecting natural autoantibodies from destruction during heat treatment.

6. The method of claim 5 wherein the biological fluid is further subjected to heat treatment.

7. The method of claim 6 wherein the biological fluid is diluted in a buffer containing proteins and are additionally treated with iron-containing oxidizer prior to heat treatment.

8. The method of claim 1 wherein for each tested specimen of biological fluid is used control solid phase of physical sorption and the number of natural autoantibodies is determined with the aid of a calibration curve which is plotted with the use of monoclonal and polyclonal antibodies to antigen.
